(19) United States
(12) Patent Application Publication
(10) Pub. No.: US 2012/0179170 A1
(43) Pub. Date: Jul. 12, 2012

(54) Apparatus For Preventing And Correcting Pseudofolliculitis Barbae, Pseudofolliculitis Pubis, And Pseudofolliculitis Nuchae

(76) Inventor: Jesse James Payne, Margate, FL (US)

(21) Appl. No.: 13/004,891

(22) Filed: Jan. 12, 2011

(51) Int. Cl. A61B 17/50

(52) U.S. Cl. 606/133

(57) ABSTRACT

The present invention discloses a device for physically dislodging, extricating, and manipulating ingrown body and facial hair so as to prevent and correct pseudofolliculitis barbae, pseudofolliculitis pubis, and pseudofolliculitis nuchae. The device comprises a rasp, an insert, a handle, and a cap. The rasp is the primary component of the device. The rasp extricates ingrown hair from hair follicles; dislodges imbedded hair from underneath the epidermal layer of the skin thereby allowing the hair to fully emerge from the follicle.

The rasp also prevents extrafollicular and transfollicular penetration of the follicle wall.

### BACKGROUND OF THE INVENTION

1. Field of the Invention

The present invention relates to a hair device, and more particularly to a hair device that physically dislodges, extricates and manipulates facial and body hair so as to prevent and correct pseudofolliculitis barbae, pseudofolliculitis pubis, and pseudofolliculitis nuchae.

2. Description of Related Art

Pseudofolliculitis barbae (PFB) or shaving bumps is a foreign body inflammatory reaction involving papules and pustules. It is a common dermatologic condition principally affecting adult men who have naturally coarse or tightly curling hair, particularly those who shave closely on a regular basis. The leading edge of closely cropped facial hair re-entering the epidermis of the skin or transecting the wall of the hair follicle results in localized inflammatory reactions over the affected site. The process can lead to secondary skin infections and, in severe cases, permanent scarring. Both sexes who have naturally coarse or tightly curling hair can develop pseudofolliculitis pubis (PFP) and pseudofolliculitis nuchae (PFN); the pathogenesis of both is similar to that of PFB.

Existing treatment and control for the ingrown hair and pseudofolliculitis consists of topical chemical applications and epilation.

The use of chemical applications includes chemical depilatories, working by breaking the disulfide bonds in hair, such as barium sulfide power depilatories, calcium thioglycolate preparation. The chemical depilatories are time-consuming to use and they are harsh and irritating on the skin, consequently, frequent usage for maintaining a clean shave is not possible without harming the skin. The use of topical retinoic acid to remove the thin covering of epidermis has to be terminated in most cases due to irritation encountered.

Epilation ranges from crude manual plucking, to electrolysis, and surgical care. In most cases, manual hair plucking or epilator does not completely remove the hair, thereby allowing the remaining stub to penetrate the follicular wall leading to exacerbation of the disease. Electrolysis and surgical removal of hair are too costly for many people who have pseudofolliculitis. Electrolysis is also painful and requires multiple treatments. The potential problems with most laser and high-intensity light source hair removal modalities are depigmentation complications and permanent hair loss. For many men who have pseudofolliculitis barbae (PFB), facial hair is valued both as a rite of passage into manhood and for personal grooming and appearance. Electrolysis or laser or high-intensity light source hair removal modalities apparently are not the preferred choice for these men.

Others use a razor with a single blade or special wire-wrapped blade to avoid shaving too closely. One company produces a blade razor with a spacer over the blade to prevent it from cutting too close. This may work for some patrons; however, it still irritated existing bumps and did not stop further bumping.

U.S. Pat. No. 6,402,763 to Scott (2002) discloses a device rotatably extricating the non-rooted end of the hair from the layers of skin in which it has penetrated comprising a housing and a blade member disposed in the housing. U.S. Pat. No. 4,709,481 to Moore (1987) discloses an instrument for use in extricating an ingrown hair from the skin, the instrument comprising an elongate handle and a tip attached to the handle, wherein the tip comprises a curved and tapered blade. The aforementioned devices in these two patents deal with one ingrown hair at a time; thus they are time consuming to use.

U.S. Pat. No. 4,151,644 to Locke (1979) discloses a device for lifting and dislodging ingrown facial or neck hair from the skin either prior to or during the act of shaving. The implement for lifting and dislodging ingrown hair is provided with a plurality of spaced rectangular-shaped raking teeth. U.S. Pat. No. 4,741,103 to Hultman (1988) discloses a razor having a knurled guard bar and a single sharp blade particularly adopted for shaving one suffering from PFB. The aforementioned devices cannot be used independently from a shaving operation because the conditioning features are in close proximity to the cutting edges.

U.S. Pat. No. 4,325,392 to Iten et al. (1982) discloses a brush having bristles of varying heights in predetermined configurations for extirpating ingrown facial hair prior to shaving. The bristles are easy to break; it has a short lifetime. EP 0761393 B1 to Schächter (2003) discloses devices used for the conditioning treatment of the skin and/or the hairs, especially in the region of the human face and especially prior to a shaving operation; said device comprises profile features as steps, ridges, or file-type or rasp-type teeth or calottes or other small projections which are provided on at least a part of the surface of the device; said device also serve to protect the razor blade edges. The objective of Schachter's invention is to lift up the hair of the beard and enable the razor blade edge to cut the hair close to the skin surface during said subsequent shaving act. The conditioning features on the device that contact with the skin are narrow resulting in inefficient conditioning of the skin and the hair.

It is clear that the aforementioned methods or products of treating PFB, PFP, and PFN are not satisfactory solutions for the adult men or women who have naturally coarse or tightly curling hair, particularly those who shave closely on a regular basis. The present invention is a great invention over existing products and techniques for addressing the causes and conditions of PFB, PFP, and PFN.

### SUMMARY OF THE INVENTION

The object of the present invention is to provide a mechanical device that can prevent and correct PFB, PFP, and PFN.

The object of the present invention is to prevent and correct the problems of PFB, PFP, and PFN by extricating ingrown hair from within the hair follicle and dislodging imbedded hair from the underneath the epidermal layer of the skin.

The object of the present invention is to prevent PFB, PFP, and PFN by uncurling and relocating coarse curly hair away from the hair follicle before a person commences to shave or clip their facial, pubic, or other body hair, or removing freshly clipped or shaved hair away from the hair follicle, inhibiting it from burrowing under the epidermal layer of the skin.

A further object of the present invention is to provide a device that is easy to use and carry, time-saving, simple to manufacture, low production cost and does not cause skin irritation.

The present invention is a durable mechanical implement comprising an insert, a handle, a cap, and a rasp, that physically dislodges and extricates ingrown hair facial and body hair as well as manipulates it away from the hair follicle so as to prevent and correct PFB, PFP, and PFN. The rasp is the primary component of the present invention. It is a sheet of metal, ergonomically designed to match the curvature of the hairy regions of the human face, neck, and pelvic regions, having a plurality of spaced protrusions on the metal sheet for extricating and dislodging ingrown hair. It dislodges ingrown hair trapped underneath the thin covering of epidermis that inhibits the hair from emerging from the follicle. It uncurls and relocates coarse curly hair away from the hair follicle before and after a shave or clip of facial, pubic, or other body hair.

The rasp corrects the conditions of PFB, PFP, and PFN whether the conditions are caused by extrafollicular or transfollicular penetration of the hair follicle. It accomplishes this by extricating ingrown hair from the hair follicle and dislodging hair imbedded underneath the epidermal layer of the skin by gently seizing and freeing ingrown or imbedded hair without completely plucking it from the follicle. The rasp also prevents extrafollicular and transfollicular penetration of the hair follicle. Extrafollicular penetration occurs when a curly hair reenters the skin. Transfollicular penetration occurs when the sharp tip of a freshly shaved or clipped hair pierces the follicle wall. The rasp prevents both types of penetration as it seizes, and relocates the sharp tip of the hair away from the follicle thereby preventing the hair from either imbedding itself underneath the epidermis or hooking back into the follicle.

The insert is fused into the top of the handle and provides the base and platform on which the rasp is mounted. The handle is ergonomically designed to fit the human hand. In the preferred embodiment, it is round on the rasp side and is flat on the side opposite the rasp. This facilitates the user by providing grip and control of the instrument. The cap is used to cover the rasp component of the appliance. It has a clip on the side which adds a visual aesthetic and facilitates in ease of transport and access to the instrument. The cap is also half-round to conform and complement the symmetry of the handle. The insert, handle, and cap are made of injection molded plastic.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a perspective view of the preferred embodiment of the device in a closed position according to the present invention.

FIG. 2 is a perspective view of the preferred embodiment of the device in an open (working) position according to the present invention.

FIG. 3 is an exploded view of the preferred embodiment of the device according to the present invention.

FIG. 4A shows a front view of the preferred embodiment of the rasp according to the present invention.

FIG. 4B shows a side view of the preferred embodiment of the rasp according to the present invention.

FIG. 4C shows a top view of the preferred embodiment of the rasp according to the present invention.

FIG. 4D shows an expanded view of a partial rasp of FIG. 4A.

FIG. 5 shows a side view of the preferred embodiment of the insert according to the present invention.

FIG. 6 shows a side view of the preferred embodiment of the handle according to the present invention.

FIG. 7A shows a front view of the preferred embodiment of the cap according to the present invention.

FIG. 7B shows a side view of the preferred embodiment of the cap according to the present invention.

### DESCRIPTION OF THE PREFERRED EMBODIMENT

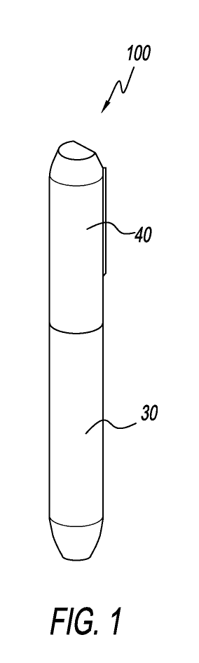

Referring to FIG. 1, there is disclosed an embodiment of device 100 according to the present invention. The device is useful in preventing and correcting pseudofolliculitis barbae, pseudofolliculitis pubis, and pseudofolliculitis nuchae by extricating ingrown hair from the hair follicle and dislodging imbedded hair from the underneath of the epidermal layer of the skin. The invention comprises a rasp, an insert, a handle, and a cap.

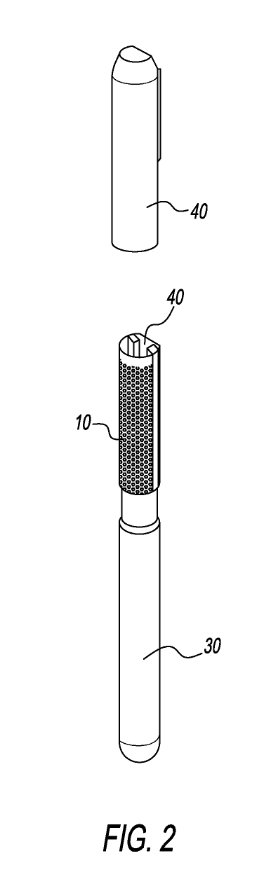

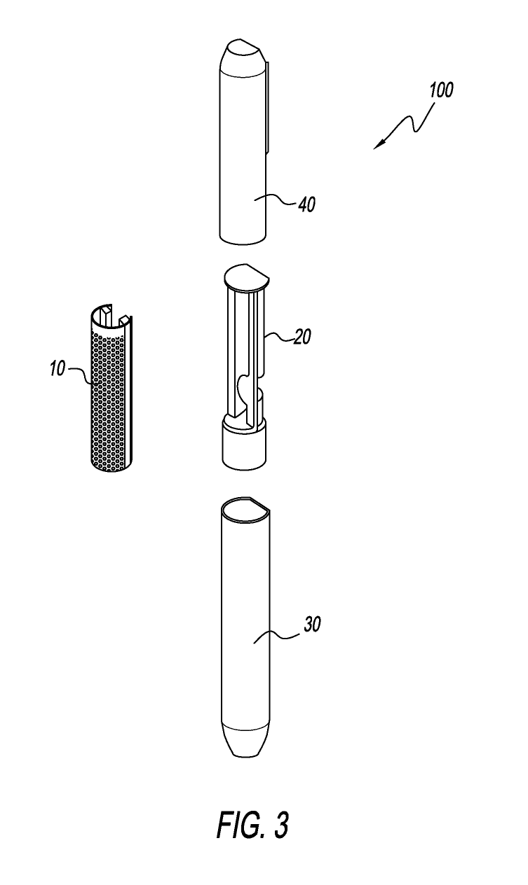

FIG. 1 shows a perspective view of a preferred embodiment of the device in a closed position wherein only the handle 30 and the cap 40 are observed; the rasp 10 and insert 20 is enclosed by the cap 40. While FIG. 2 shows the device in an open (working) position where the bottom of the insert 20 is fused into the handle 30; the rasp 10 is mounted onto the insert 20, and the cap 40 is taken off from the rasp/insert 10/20. An exploded view of the preferred embodiment of the device was disclosed in FIG. 3 where the cap 40 is taken off from the insert 20 and rasp 10; the insert 20 is not fused into the handle 30 and the rasp 10 is removed from the insert 20.

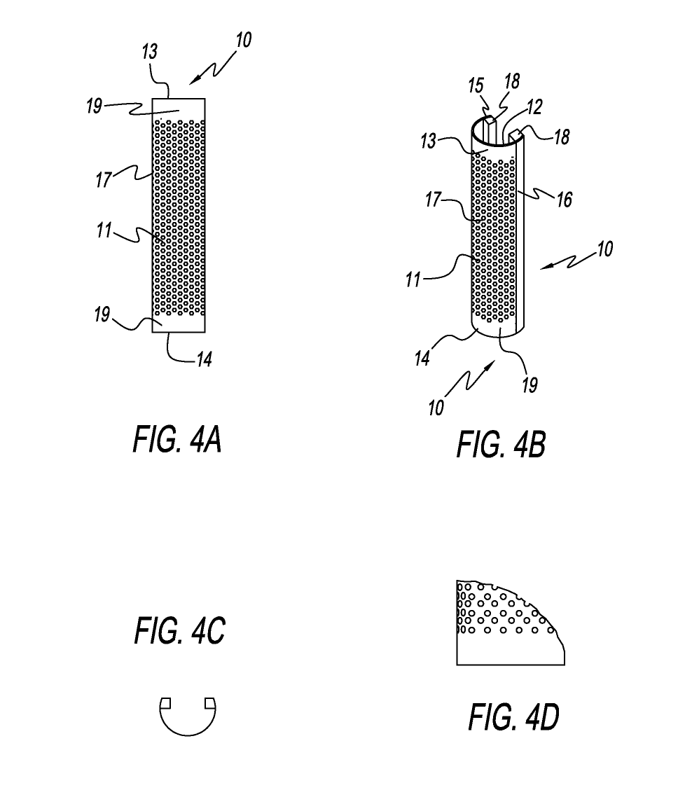

Referring to FIG. 4, there is disclosed a preferred embodiment of the rasp 10 according to the present invention. The rasp 10 is the primary component of the present invention. The rasp 10 extricates ingrown hair from hair follicles; dislodges imbedded hair from underneath the epidermal layer of the skin. The rasp 10 also prevents extrafollicular and transfollicular penetration. It accomplishes this by dislodging and displacing hair away from the hair follicle and uncurling the hair to prevent the hair from imbedding itself underneath the epidermal layer of the skin.

The rasp 10 is ergonomically designed to match the curvature of the hairy regions of human face, neck, and pelvic region. FIGS. 4A and 4B illustrate the front view and the side view of the rasp, respectively. In general, the rasp is a piece of a curved elongated metal sheet having a concave outer surface 11 and an inner surface 12 as well as a top edge 13, a bottom edge 14, a first side edge 15 and a second side edge 16. Rasp has a plurality of protrusions 17 on the concave outer surface. Along each of the two side edges on the inner surface, the rasp has a projection 18 to be snapped onto the insert 20. It can be removed for cleaning and sanitizing. The protrusions 17 are evenly distributed over the entire rasp 10 except the four border region 19 along the four edges. In addition to the semi-circle shape, the rasp 10 may be of other different shapes. Besides having only one curvature, the rasp 10 may have two different curvatures on the metal sheet.

FIG. 4A, FIG. 4C, and FIG. 4D disclose the preferred embodiment with detailed dimensions. FIG. 4A shows the front view of the rasp, FIG. 4C shows a top view of the rasp; and FIG. 4D shows an expanded view of a partial rasp of FIG. 4A. The preferred embodiment of the rasp is a piece of elongated curved metal sheet having 2 inches in height, 0.442 inch in width, 0.236 inch in radius, and 0.006 inch in thickness; the border region is within about 0.16 inch of each edge; all the protrusions 17 are of the same size with a diameter of 0.025 inch; the projection 18 rectangular prism is 0.083×0.083×2 inches in width, length and height. The rasp including all components may vary in size, and curvature. The rasp 10 is made of stainless steel or another hard and moldable non-ferrous metal that can be fabricated into a rasp 10. The protrusion 17 may be of different shapes and dimensions.

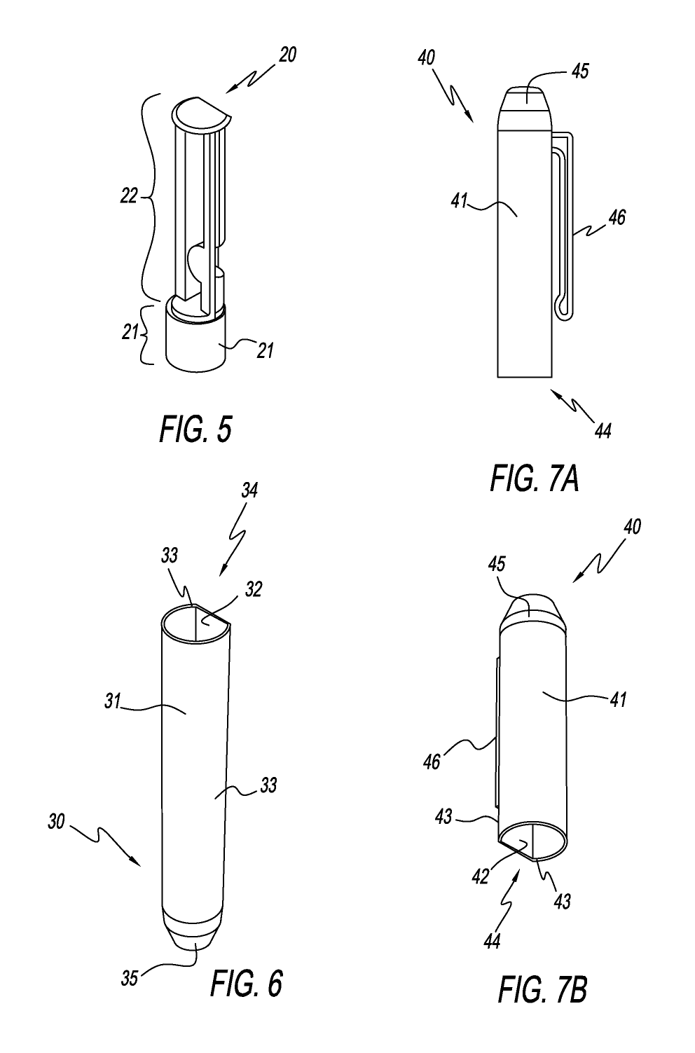

Referring to FIG. 5, there is disclosed an embodiment of the insert 20 according to the present invention. The insert 20 has a bottom member 21, which is fused into the top of the handle 30, and a body member 22, which has a dimension adapted to match the rasp 10 in order to provide the base and platform on which the rasp 10 is mounted. The insert 20 is made of injection molded plastic.

The handle 30 disclosed in FIG. 6 is ergonomically designed to fit the human hand. The handle 30 has two sides; the round side 31 being on the same side of the concave surface 11 of the rasp 10, the flat side 32 being opposite to the concave surface 11 of the rasp 10. The round side 31 and the flat side 32 connect with each other along the longitudinal edges 33 to form a hollow half round D-shaped elongated member. The top 34 of the handle 30 is open for receiving the bottom 21 of the insert 20. The bottom 21 of the insert 20 is fused into the top 34 of the handle 30. The bottom 35 of the handle 30 is closed. The D-shaped configuration of the handle 30 facilitates the user by providing grip and control of the device. In the preferred embodiment, the height of the handle 30 is about 3.375 inch. The largest width of the cross section of the top opening 34 is about 0.491 inch. The angle between the round side 31 and the flat side 32 is approximately 89.5 degrees. The thickness of the handle 30 is about 0.031 inch. Around the lower end, the handle 30 gradually forms a dome at the bottom tip 35. The handle 30 may vary in size and curvature. The handle 30 can be made of injection molded plastic. Besides a D-shape configuration, the handle 30 may be of other shapes.

Referring to FIGS. 7 A and 7B, there is disclosed a preferred embodiment of the cap 40 according to the present invention. The cap 40 is made of injection molded plastic and is used to cover the rasp and the insert of the device. The cap 40 has two sides; the round side 41 being on the same side of the concave surface 11 of the rasp 10, the flat side 42 being opposite to the concave surface 11 of the rasp 10. The round side 41 and the flat side 42 connect with each other along the longitudinal edges 43 to form a hollow half round D-shaped elongated member. The bottom entrance 44 of the cap 40 is open for receiving the rasp 10 and insert 20. The top 45 of the cap 40 is closed. The D-shaped configuration of the top 40 is to conform and complement the symmetry of the handle 30. The cap has a clip 46 on the flat side 42 which adds a visual aesthetic and facilitates in ease of transport and access to the instrument. The clip 46 may contain a magnet or be made of a magnetic material so that it can be attached to a metal piece for easy storage or access. The clip 46 may be used to hang on a pocket for easy access. In the preferred embodiment, the height of the cap 40 is about 2.500 inch; the largest width is about 0.494 inch; the thickness is about 0.031 inch. The length of clip 46 is about 1.600 inch. The cap 40 gradually slopped upwards and inwards and close at the top. The cap 40 may vary in size and curvature. The cap 40 can be made of injection molded plastic. In addition to the D-shape configuration, the cap 40 may be of other shapes.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

1. A device to prevent and correct pseudofolliculitis related conditions comprising:
(a) a rasp comprises a piece of convex half circular cylindrical elongated metal sheet, which has a outer surface and an inner surface; a plurality of spaced protrusions on said outer surface metal sheet; and a pair of projections on said inner surface pointing towards each other;
(b) a convex half circular cylindrical and half-flat insert comprises a bottom member and a body member, said body member is a base and platform for said rasp, said projections on said rasp mount on said body member;
(c) a convex half circular cylindrical and half-flat handle comprises a hollow elongated member, said bottom of said insert fused into the top of said handle; and
(d) a convex half circular cylindrical and half-flat cap comprises a hollow elongated member, said bottom of said cap receiving said rasp mounted insert.
wherein said rasp is configured with a radius of 0.2 to 0.3 inches.

2. A device to prevent and correct pseudofolliculitis related conditions comprising:
(a) a convex half-cylindrical rasp comprises a piece of convex half-round elongated metal sheet, which has a concaved ergonomically configured outer surface and a inner surface; a plurality of spaced protrusions on said metal sheet; and a pair of projections on said inner surface pointing towards each other;
(b) a convex half-cylindrical-half flat an insert comprises a bottom member and a body member, said body member is a base and platform for said rasp, said projections on said rasp mount on said body member;
(c) a handle comprises a round side and a flat side to form a hollow half-round elongated member, said bottom of said insert fused into the top of said handle; and
(d) a cap comprises a round side and a flat side to form a hollow half-round elongated member, said bottom of said cap receiving said rasp mounted insert.

3. The device of claim 1 or 2, wherein said metal sheet of rasp is stainless steel.

4. The device of claim 1 or 2, wherein said metal sheet of rasp is hard and moldable non-ferrous metal that can be fabricated into a rasp.

5. The device of claim 1 or 2, wherein said rasp is ergonomically designed to match the curvature of the hairy regions of human face, neck, and pelvic region.

6. The device of claim 1 or 2, wherein said rasp can be detached from said insert for cleaning and sanitizing.

7. The device of claim 1 or 2, wherein said insert, said handle, and said cap are made of injection molded plastic.

8. The device of claim 1 or 2, wherein said cap further has a clip on the side.

9. The device of claim 1 or 2, wherein said handle and cap are ergonomically designed to fit the human hand.

10. The device of claim 1 or 2, wherein said protrusions on said rasp have different shapes and sizes.

11. The device of claim 1 or 2, wherein the diameter of said protrusion is about 0.025 inch.
